(19) United States
(12) Patent Application Publication
(10) Pub. No.: US 2026/0315216 A1
(43) Pub. Date: Oct. 15, 2026

(54) SYSTEMS & METHODS FOR CREATING PERSONALIZED AORTIC HEART VALVES

(71) Applicant: Oregon Health & Science University, Portland, OR (US)

(72) Inventors: Gary Michael Silberbach, Portland, OR (US); Ashok Muralidaran, Portland, OR (US); Jamie You, Portland, OR (US); Matthew Dally, Portland, OR (US)

(21) Appl. No.: 19/465,526

(22) Filed: Jan. 30, 2026

Related U.S. Application Data
(60) Provisional application No. 63/751,790, filed on Jan. 30, 2025.

(51) Int. Cl. A61F 2/24; A61L 27/18; A61L 27/36; G06T 7/00; G06T 7/11

(52) U.S. Cl. CPC A61F 2/2415; A61L 27/18; A61L 27/3604; A61L 27/3679; G06T 7/0012; G06T 7/11; A61F 2240/002; A61F 2240/005; A61L 2430/20; G06T 2207/10081; G06T 2207/10088; G06T 2207/10136; G06T 2207/30048

(57) ABSTRACT

Systems and methods are disclosed for creating a customized prosthetic heart valve based on individual-specific heart valve anatomy. The disclosed systems and methods enable the creation of prosthetic heart valves that are derived from the three-dimensional geometry of an individual's aortic sinuses. The methods may also be used to create other prosthetic heart valves or to prepare heart valve leaflets which may be sutured into a vessel wall or delivered percutaneously by an operator in a cardiac catheterization laboratory.

### TECHNICAL FIELD

This disclosure relates generally to creating customized prosthetic heart valve based on individual-specific heart valve anatomy. More specifically, this disclosure enables the creation of prosthetic aortic heart valves that are derived from the three-dimensional geometry of an individual's aortic sinuses.

### BACKGROUND INFORMATION

The aortic sinus is the portion of the aorta that surrounds the aortic valve at the aortic root. In a normal, healthy aortic valve the three individual sinuses comprising the aortic sinus are roughly equal in size and shape. Moreover, each of the leaflets of a healthy aortic valve are matched to the size and shape of the sinuses that surround them. In patients with bicuspid aortic valve disease, however, aortic anatomy is often characterized by three unequally sized and spaced aortic sinuses and a heart valve in which two leaflets are formed. When these patients need to have their diseased aortic valve replaced with a prosthetic aortic valve, the current standard of care includes fitting them with a replacement prosthetic aortic valve which has three leaflets of equal size and shape. That is, the replacement valve is mismatched to the anatomic geometry of their native sinus spaces.

As an example of current standard of care for valve replacement, the Ozaki procedure has recently entered the canon of operation for the bicuspid aortic valve. During the procedure, the surgeon visually examines the shape of the native aortic valve and the intercommisural distance is measured by “Ozaki sizers” (Tokyo Research Center for Advanced Surgical Technology Co, Ltd, Tokyo, Japan). The sizers are standardized from small to large and do not take into account the asymmetry of the native valve or sinuses. Based on the measurement from the sizers, a set of new valve leaflets are cut from autologous pericardium using a standardized stencil and sewn into place to restore valve function.

The inventors have proposed that implantation of personalized aortic valve prostheses that reflect the unequal shapes of a patient's sinuses will lead to superior health outcomes owing to improved fluid mechanics as blood passes through the valve and into the aorta. U.S. Pat. No. 11,464,639 describes methods for extracting surface descriptions of the aortic sinuses from tomographic medical imaging data and using computer aided design methods to generate surface descriptions of individual leaflets that each have a 1-to-1 size and shape correspondence with its associated aortic sinus. However, there remains a need for tools and methods to reliably create patient-specific heart valve prostheses from these surface descriptions of the individual leaflets so that custom-designed heart valve replacement can be realized.

### SUMMARY OF THE DISCLOSURE

Disclosed herein are systems and methods that enable the creation of personalized prosthetic heart valves. By tailoring the geometry of the prosthetic leaflets and anchoring frame (also called an armature) to an individual's own anatomy, the heart valves produced by the disclosed systems and methods exhibit improved hemodynamic valve function compared to currently available prosthetic valves.

Provided is a method of making and using a templating system configured to facilitate creation of a set of custom replacement aortic valve leaflets. The templating system includes a set of three separate templating forms, with each templating form configured to be used to create an individual and distinct replacement leaflet whose shape is derived from one of the three sinuses. Each templating form is comprised of two companion parts: a first template form presenting a concave surface that corresponds to a replacement leaflet shape to be created, and a second template form presenting a convex surface that corresponds to the same leaflet shape to be created. The concave and convex surfaces of these template forms are matably congruent so that, when brought together with a piece of material between them (such as a sheet of pericardium or a biocompatible fabric), the material is sandwiched between the two surfaces in a secure manner. With the material thusly constrained between the concave and convex surfaces, it is possible to trim away the material extending outwardly from the mating to surfaces to create a leaflet of the desired shape. The template forms, when used in this manner, allow the user to create precisely sized and shaped replacement leaflets in a highly reproducible fashion.

Also provided is method for creating a replacement aortic valve prosthesis that is specifically tailored to an individual's native aortic sinus anatomy. Creation of such a replacement valve entails receiving a three-dimensional anatomical imaging data set that spans the aortic sinus at the aortic root and segmenting, using computer-aided design tools, a surface representation of the aortic sinus anatomy. For each of the three aortic sinuses, a surface representation of a replacement leaflet shape is defined based on the sinus geometry and used to create a pair of matably congruent template forms using an additive manufacturing approach (i.e., 3-D printing) or other manufacturing techniques known in the art. A custom anchoring frame, or armature, is also designed and sized based on the individual-specific anatomical imaging data and shapes of the replacement leaflets. The custom-sized armature may also be fabricated using additive manufacturing or other manufacturing techniques known in the art. The leaflets may then be joined to the armature to form a replacement aortic valve prosthesis that is personalized to the individual's unique anatomy.

Additional aspects and advantages will be apparent from the following detailed description of preferred embodiments, which proceeds with reference to the accompanying drawings.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

To easily identify the discussion of any particular element or act, the most significant digit or digits in a reference number refer to the figure number in which that element is first introduced.

FIG. 1 is a photograph showing an exemplary set of three templating blocks positioned around an anchoring frame/armature construct.

FIG. 2A shows an example set of components for a leaflet templating system and a substantially flat piece of material that is to be formed into a leaflet.

FIG. 2B shows an example set of components for a leaflet templating system and a piece of material draped into the concave surface of one component of the leaflet templating system.

FIG. 2C shows an example set of components for a leaflet templating system with piece of material sandwiched between the concave surface of a concave template form and the convex surface of convex template form.

FIG. 3A shows a computer model of asymmetric aortic sinuses as segmented from a set tomographic images spanning the aortic root of a patient. A set of three planes are shown intersecting the model to define the boundaries of three sinuses from which three leaflet geometries are defined.

FIG. 3B shows an example of a set of sinus-matched bovine tissue leaflets derived from the computer model of FIG. 3A attached to a 3D printed armature to form a prosthetic valve in the open position.

FIG. 3C shows an example of the prosthetic valve of FIG. 3B as sutured into a 3D printed aortic sinus as viewed from beneath with the valve leaflets in the closed position.

FIG. 4 is a photo showing an example of a prosthetic heart valve sewn into a 3D-printed flexible resin model of an aortic sinus.

### DETAILED DESCRIPTION OF EMBODIMENTS

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate discussion and are not intended to restrict the application of embodiments of the present invention.

The terms “coupled” and “connected,” along with their derivatives, may be used. It should be understood that these terms are not intended as synonyms for each other. Rather, in particular embodiments, “connected” may be used to indicate that two or more elements are in direct physical contact with each other. “Coupled” may mean that two or more elements are in direct physical or electrical contact. However, “coupled” may also mean that two or more elements are not in direct contact with each other, but yet still cooperate or interact with each other.

For the purposes of the present invention, the phrase “A/B” means A or B. For the purposes of the present invention, the phrase “A and/or B” means “(A), (B), or (A and B)”. For the purposes of the present invention, the phrase “at least one of A, B, and C” means “(A), (B), (C), (A and B), (A and C), (B and C), or (A, B and C)”. For the purposes of the present invention, the phrase “(A)B” means “(B) or (AB)” that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Unless otherwise noted or explained, all technical and scientific terms used herein are used according to conventional usage and have the same meaning as commonly understood by one of ordinary skill in the art which the disclosure belongs. Although methods, systems, and apparatuses/materials similar or equivalent to those described herein can be used in the practice or testing of the present disclosure, suitable methods, systems, and apparatuses/materials are described below.

Disclosed herein are systems and methods that enable the creation of personalized prosthetic heart valve leaflets and heart valves. In a typical application, the systems and methods include the receipt of three-dimensional tomographic imaging data from which individual-specific heart valve anatomy is extracted. In a specific application, the three-dimensional anatomy of the aortic sinus (the part of the aorta that surrounds the aortic valve) is used as a basis for generating a set of individually tailored prosthetic valve leaflets for use in a prosthetic valve. Such individually tailored prosthetic valve leaflets and prosthetic valves that are created based on the geometry of an associated sinus are referred to herein as “sinus-matched leaflets” and “sinus-matched valves.” In some applications, a custom anchoring frame, or armature, may also be derived from the set of individually tailored prosthetic valve leaflets for use in construction of a sinus-matched prosthetic aortic valve.

In a normal healthy aortic valve, the sinuses are equally sized and spaced and each of the leaflets comprising the valve are matched in size and shape to their respective sinuses. That is, the healthy aortic valve and sinuses are largely symmetrical. Patients who have bicuspid aortic valve disease, however, typically have three aortic sinuses that are of unequal size and shape (i.e., the sinuses are asymmetric). These patients often need to have their aortic valve replaced as the valve stenoses over time and function becomes compromised. The inventors have proposed that personalized aortic valve prosthetics that match the unequal shapes of these patients'sinuses-as opposed to replacement valves with a more symmetric configuration that does not match their native sinus geometry-will have superior health outcomes.

Disclosed herein are systems and methods which operationalize an approach for creating a sinus-matched aortic valve using patient-specific three-dimensional surface representations of a patient's own aortic sinuses. A method for creating three-dimensional surface representations of a patient's own aortic sinuses from three-dimensional tomographic imaging data is described in detail in U.S. Pat. No. 11,464,639, which is incorporated by reference herein in its entirety. The present disclosure builds upon and extends this method by describing a system and methods for creating an individual-specific prosthetic aortic valve that is geometrically matched to the native sinus anatomy.

It is understood that the methods, aortic valves, leaflets, prosthetics, and other aspects described herein are included for use in mammalian patients or subjects. In some embodiments, they are included for use in human patients or subjects. In some embodiments they are intended for use in infants, children, adolescents, and adults.

In some embodiments, the aortic valve prosthesis described above is a bioprosthesis. In some embodiments, the bioprosthesis comprises porcine tissue. In other embodiments, the bioprosthesis comprises bovine tissue. In still other embodiments, the bioprosthesis comprises human tissue.

The terms “bioprosthesis” and “bioprosthetic” as used herein refers to a prosthetic device comprising an animal part or an animal tissue, including porcine, bovine, or human parts or tissues. In particular, they refer to sheets of natural tissue used to form any portion of a prosthetic device, including a framework or leaflets (cusps) for cardiac valve repair or replacement.

The terms “imaging” or “medical imaging” or “anatomical imaging” or “tomographic imaging” herein refer to techniques and process used to create visual representations, either in the form of two-dimensional image data (images) or three-dimensional image data (image stacks), of the interior of a subject's body for clinical analysis, diagnosis, or medical treatment. Examples of medical imaging useful for the present uses include Magnetic Resonance Imaging (MRI), cardiac MRI, computed tomography (CT)-angiography, or three-dimensional echocardiography.

As described in greater detail below, an aortic valve templating system that includes a leaflet templating system is configured to create a set of tool forms for shaping and sizing a new set of patient-specific valve leaflets for deployment in a replacement aortic valve. The aortic valve templating system and leaflet templating system are intended to be custom manufactured, for example by 3D print manufacturing techniques known in the art, so that the geometry of the replacement aortic valve components (i.e., replacement leaflets and armature) are specifically tailored to the native sinus anatomy of the valve recipient.

The sinus-matched valve templating system disclosed herein comprises three separate templating forms corresponding to each of the three aortic sinuses. Each of these templating forms is made up of two separable mating parts: a concave template form and a convex template form. The concave template form includes a concave surface feature having dimensions that correspond to those of a replacement leaflet shape derived from the anatomical shape of an individual aortic sinus as extracted from patient-specific 3D imaging data. Similarly, the convex template form includes a convex surface feature having dimensions that correspond to those of the same replacement leaflet shape derived from the same individual aortic sinus anatomy. Accordingly, these concave and convex surface features are congruent surfaces having matching shapes and curvatures. The curvilinear surface of the concave template form represents the sinus-facing side of a valve leaflet to be produced, while the curvilinear surface of the companion convex template form corresponds to the coaptation side of the leaflet to be produced.

The companion concave template form and convex template forms are said to be “matably congruent.” As used herein, the term “matably congruent” refers to the design of two or more surfaces on separate parts that match when brought together, such as a first convex surface and a second concave surface that may be brought together with a material between them such that the material is held in a desired three-dimensional shape or arrangement between the two surfaces.

As will be described in an example below, the two separable, matably congruent parts of a given templating form-the concave template form and the convex template form-are intended to be pressed together with a piece of fabric or tissue sandwiched between the concave and convex mating surfaces to facilitate precise forming and trimming of the fabric or tissue into the desired leaflet shape. Once the trimmed leaflet is released it can be mounted onto an anchoring frame with two other custom leaflets prepared in the same manner to form a prosthetic aortic valve. Alternatively, the custom leaflets themselves may be sutured directly into sinus space of the patient aortic root to effect valve replacement.

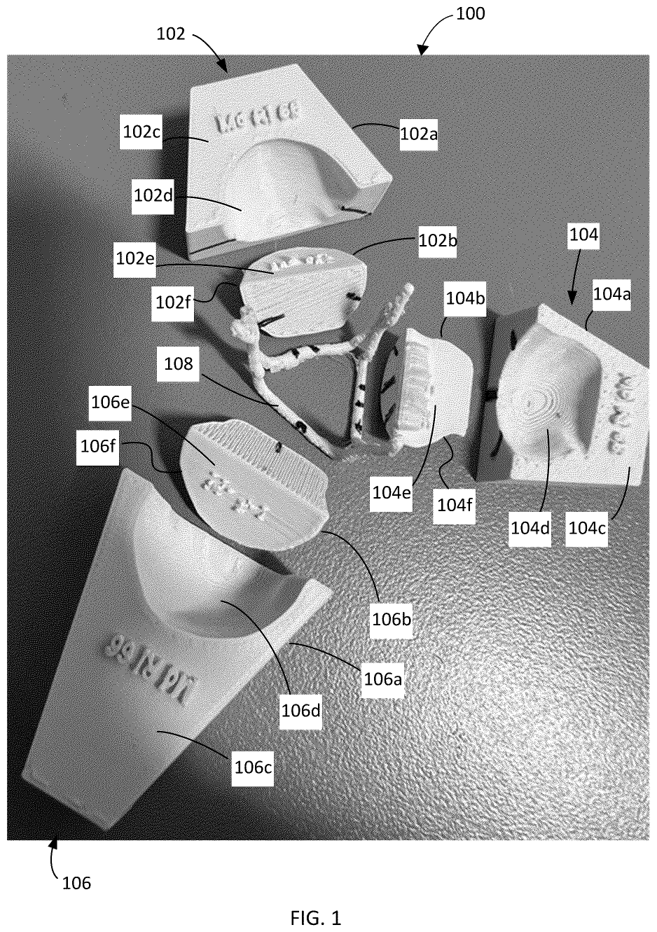

FIG. 1 shows a photo depicting an exemplary embodiment of aortic valve templating system 100. The concave and convex template forms depicted in FIG. 1. are configured roughly in the shape of solid blocks with matably congruent surfaces. Accordingly, for the components of templating system 100 depicted in FIG. 1, the nomenclature “templating block” and “concave (or convex) template block” will be adopted to refer to the depicted templating form and concave (or convex) template forms, respectively.

Returning to FIG. 1, templating system 100 comprises a first templating block 102, a second templating block 104, a third templating block 106, and an anchoring frame/armature 108. Together, first templating block 102, second templating block 104, and third templating block 106 make up leaflet templating system. First templating block 102 comprises a first concave template block 102a and first convex template block 102b. First concave template block 102a includes an extruded body portion 102c formed about and extending away from a concave surface 102d. First convex template block 102b includes an extruded body portion 102e extending away from a convex surface 102f. Similarly, second concave template block 104a includes an extruded body portion 104c formed about and extending away from a concave surface 104d, and second convex template block 104b includes an extruded body portion 104e extending away from a convex surface 104f. Likewise, third concave template block 106a includes an extruded body portion 106c formed about and extending away from a concave surface 106d, and third convex template block 106b includes an extruded body portion 106e extending away from a convex surface 106f. It can be appreciated in the photo that each of the companion concave and convex template blocks (i.e, parts 102a and 102b; parts 104a and 104b; and parts 106a and 106b) are matably congruent at their respective concave and convex surfaces (i.e., surfaces 102d and 102f; surfaces 104d and 104f; and surfaces 106d and 106f). It can also be appreciated that the mating surfaces of the three templating blocks 102, 104, and 106 differ in size and shape to mimic the differing anatomy of the three anatomic aortic sinuses. It is also noted that the anchoring frame/armature 108 is also individual-specific (i.e., derived from the geometry of the patient aortic sinus and valve anatomy). All components shown in FIG. 1 were made from resin using a 3D printing process. However, these components may also be formed or manufactured using other methods known in the art.

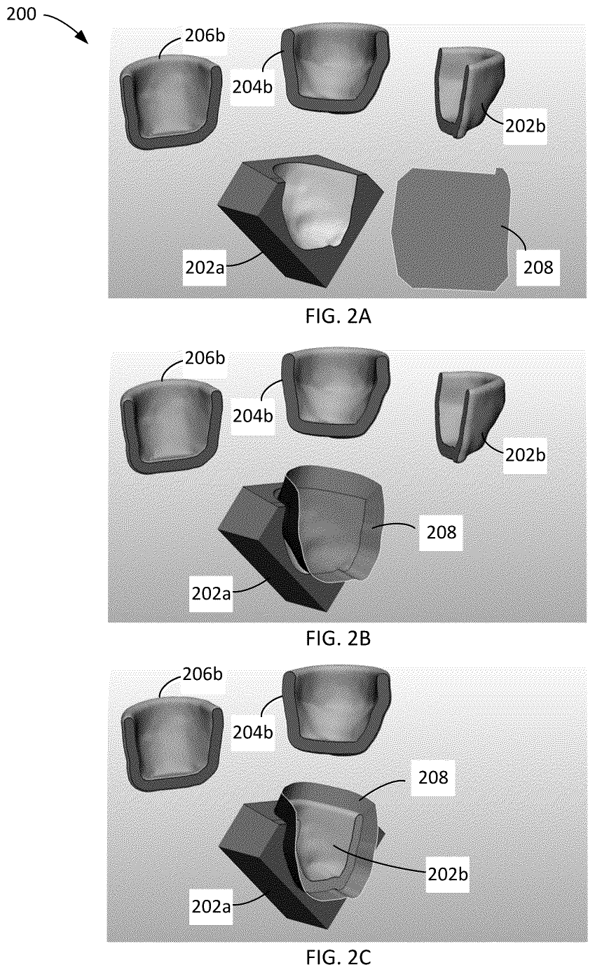

FIGS. 2A, 2B, and 2C show several components from a leaflet templating system 200 that make up aortic valve templating system 100. Together, FIGS. 2A, 2B, and 2C depict an example of a sequence of steps using various components of leaflet templating system 200 to generate a replacement leaflet from a small sheet of material, such as a piece of bovine pericardium or a synthetic or tissue engineered fabric.

FIG. 2A shows a single concave template form 202a, three convex template forms 202b, 204b, 206b, and a substantially flat sheet of material 208. In FIG. 2B, flat sheet of material 208 is shown being placed or draped into the concave surface of the concave template form 202a. In FIG. 2C, the convex surface of convex template form 202b is shown being pressed into the concave surface of concave template form 202a, thereby sandwiching sheet of material 208 between the matably congruent template forms.

Sandwiching fabric or tissue between two templates, concave template form 202a and convex template form 202b, functions as a composite molding allowing the surgeon or technician greater stability and precision to trim away excess of material (not depicted). Once released from the sandwiching templates the replacement valve leaflet matching the patient's own aortic sinus anatomy is affixed to an armature or sutured directly into the wall of the aortic sinus. It is noted that the armature approach would be amenable to interventional cardiac catheterization where the prosthetic valve attached to the armature is then delivered percutaneously, a medical procedure known as the TAVR (Transcatheter Aortic Valve Replacement) that provides a minimally invasive means to replace a damaged aortic valve.

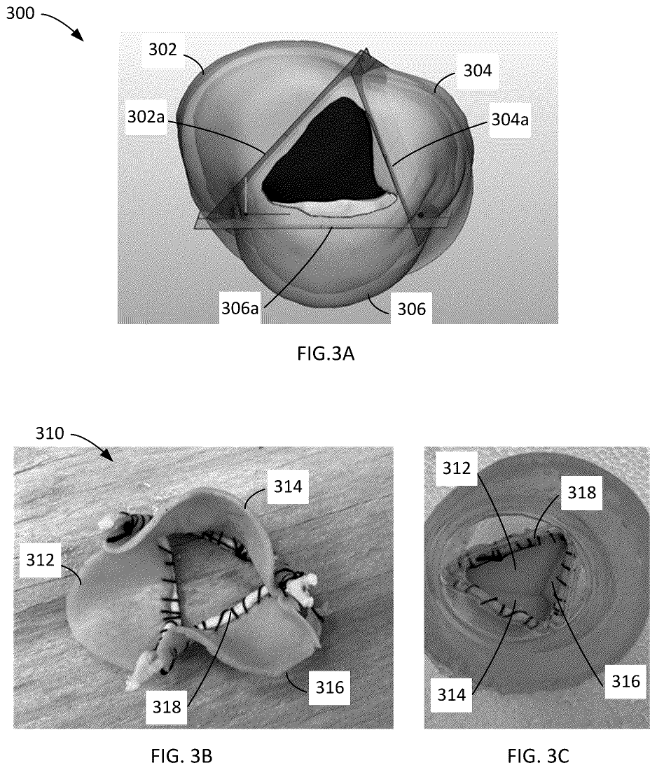

FIG. 3A shows an example of a computer model 300 of an asymmetric aortic sinus anatomy segmented from tomographic images that span the aortic root of a patient. The model is oriented so that the three sinuses 302, 304, 306 are viewed from below (i.e., from the inferior to the superior anatomic direction). A set of three construction planes 302a, 304a, and 306a are shown intersecting the model. The intersection of each of these planes 302a, 304a, and 306 with computer model 300 is used to delineate the edges of leaflet shapes derived from individual sinuses and define a set of three sinus-matched leaflet surfaces. These sinus-matched leaflet surfaces serve as templates to create the concave and convex surfaces of the templating forms (FIG. 1) to be used in the construction of a sinus-matched valve leaflets. The method of delineating and defining the geometric surface representation of a set of leaflets from a three-dimensional anatomical imaging data set is described in greater detail in U.S. Pat. No. 11,464,639, incorporated by reference herein.

FIG. 3B shows an example of a prosthetic heart valve 310 formed from a set of sinus-matched leaflets 312, 314, 316 (derived from the model depicted in FIG. 3A) affixed to an anchoring frame/armature 318 (also derived from the model depicted in FIG. 3A). FIG. 3B is a view of prosthetic heart valve 310 viewed from above with leaflets 312, 314, 316 sewn into armature 318 displayed in the open position. In such an open position in vivo the leaflets would be nestled in their respective sinuses as blood is pumped through the valve opening into the aorta. Later during the cardiac cycle when the heart relaxes, reversing blood flow would become insinuated between each of the leaflets and their respective sinuses, forcing the leaflets to collapse toward the center of the aortic valve, leaning against one another and effectively closing the valve. FIG. 3C is a view from below showing valve 310 in this closed position with valve leaflets 312, 314, 316 collapsed upon one another. Note that in FIG. 3C, the prosthetic valve of FIG. 3B has been sutured into a 3D printed aortic sinus model to illustrate its appearance in the closed position from beneath.

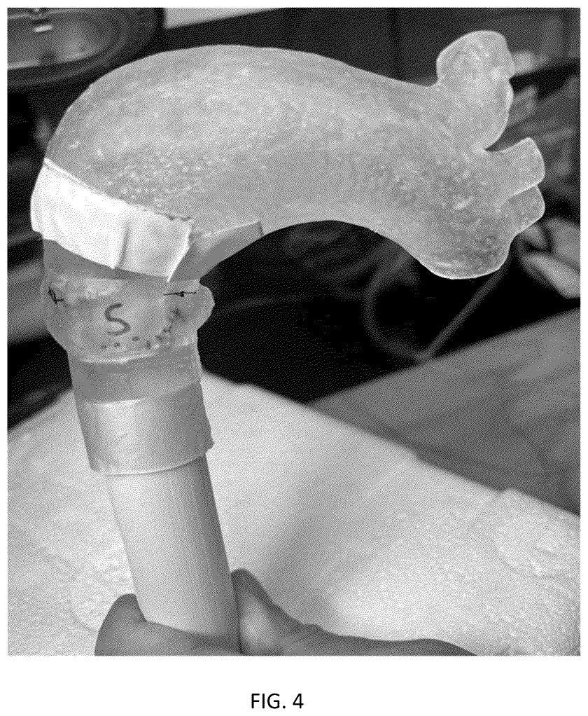

FIG. 4 shows an example of an asymmetrical valve that is sewn into an asymmetrical aortic sinus portion of an individual-specific aorta model made from 3D-printed flexible resin. Such a 3D printed, flexible resin aorta model with valve leaflets sewn into place may be deployed into a flow chamber, for example, to evaluate the fluid mechanics and fluid-structure interaction of the aortic valve & aorta assembly under pulsatile flow conditions to assess hemodynamic fitness. In more typical use cases, a surgeon or technician in the operating room could use the aortic valve templating system and associated set of templating forms to cut sinus-matched leaflets to create a prosthetic valve for surgical placement in the patient's asymmetrical aortic sinuses. Alternatively an interventional cardiologist or technician could deploy the prosthetic valve percutaneously in the cardiac catheterization laboratory.

The systems and methods for creating individual-specific sinus-matched valve leaflets described herein may utilize a wide range of materials for the construction of prosthetic valves. Leaflets can be formed from a biological material or from a synthetic material that is biocompatible, resists clotting, and has functional mechanical properties similar to native leaflets. The material comprising the leaflet can be chemically or physically treated to prevent immunological reaction, calcification, or fatigue. Biological materials used to construct leaflets may be fixed, for example, using glutaraldehyde or other fixative, or a photofixation procedure. Exemplary biological materials can include, but are not limited to human, bovine, or porcine pericardium. Human, porcine, or bovine valve leaflets are also contemplated as materials from which new leaflets may be constructed. Materials may also be selected from a variety of fabrics or textiles that can be natural substances or chemically synthesized. These textiles can be woven threads or printed using, for example, 3D printing or other additive manufacturing techniques. Tissue engineered materials may also be utilized.

Examples of such materials include those traditionally found in prosthetic heart valves such as polyethylene terephthalate (Dacron), polytetrafluoroethylene (PTFE, GoreTex), or pericardial tissue (bovine, equine, porcine, autologous). In addition, new materials such as 3-D printed fabrics or bioengineered stem cell caffold or decellularized constructs as found in tissue engineering applications may be used. A potential advantage of using tissue engineered materials such as stem cell-seeded scaffold constructs is that a prosthetic heart valve constructed in such a manner has the potential to undergo remodeling processes to enable in vivo growth and mechanical adaptation of the valve over time to accommodate changes in size of the surrounding aorta and sinuses of the valve recipient (i.e., growth & development, as in children).

It should be noted that in the typical use case the disclosed aortic valve and leaflet templating system is intended to provide individual-specific fixtures to allow creation of three custom-sized valve leaflets. However, it is contemplated that in some instances it may be desirable to generate and utilize a single leaflet templating system based on only one of the patient's aortic sinuses to allow creation of three equally sized leaflets which, when affixed to a symmetric armature, will produce a substantially symmetric prosthetic valve. In some clinical situations, such a valve may exhibit more favorable coaptation of the leaflets during closure and thereby better mitigate backflow (called aortic insufficiency). In addition, such a valve may also have utility for in vivo experimental setups to study the fluid mechanical effect and fluid-structure interaction of such a symmetric valve configuration in an anatomic model of an aorta. Such implementations and embodiments are readily enabled without undue experimentation or modification to the disclosed systems and methods described herein.

It is understood that in some embodiments the templating forms created from the methods herein could be modified without undue effort to allow for additional material in the leaflet to accommodate joining the leaflet into position in a heart valve, whether in vivo directly to tissue or to an armature. It is also understood that in some embodiments the templating forms may be fabricated to form a leaflet having a smaller surface area to accommodate the dimensions of another material or device with which it is utilized, such as a an armature or other valve replacement device or material.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

What is claimed is:

1. A templating form for use in the creation of a custom replacement aortic heart valve leaflet, comprising:
a first template form presenting a concave surface corresponding to a replacement leaflet shape; and
a second template form presenting a convex surface corresponding to the replacement leaflet shape, such that the convex surface and the concave surface are matably congruent, and wherein the replacement leaflet shape is derived from three-dimensional anatomical imaging data of an aortic sinus.

2. A method of preparing a templating form for use in the creation of a custom replacement aortic valve leaflet, comprising:
receiving a three-dimensional anatomical imaging data set that includes an aortic sinus;
segmenting from the three-dimensional anatomical imaging data set a surface representation of the aortic sinus;
defining a replacement leaflet shape based on the surface representation of the aortic sinus;
creating a first template form presenting a concave surface corresponding to the replacement leaflet shape; and
creating a second template form presenting a convex surface corresponding to the replacement leaflet shape, wherein the concave surface and the convex surface are matably congruent.

3. A custom replacement aortic valve leaflet, prepared by the steps of:
receiving a three-dimensional anatomical imaging data set that includes an aortic sinus;
segmenting from the three-dimensional anatomical imaging data set a surface representation of the aortic sinus;
defining a replacement leaflet shape based on the surface representation of the aortic sinus;
creating a first template form presenting a concave surface corresponding to the replacement leaflet shape; and
creating a second template form presenting a convex surface corresponding to the replacement leaflet shape, wherein the concave surface and the convex surface are matably congruent;
sandwiching a substantially flat piece of prosthetic leaflet material between the concave surface of the first template form and the convex surface of the second template form, such that a first portion of the prosthetic leaflet material is secured between the concave and convex surfaces, and a second portion of the prosthetic leaflet material extends beyond the concave and convex surfaces;
trimming and discarding the second portion of the prosthetic leaflet material extending beyond the concave and convex surfaces; and
separating the first template form and second template form to release the first portion of the prosthetic leaflet material, the first portion of the prosthetic leaflet material now configured in the form of a custom replacement aortic valve leaflet.

4. A replacement aortic valve prosthesis, prepared by the steps of:
receiving a three-dimensional anatomical imaging data set that includes an aortic sinus;
segmenting from the three-dimensional anatomical imaging data set a surface representation of the aortic sinus comprising three aortic sinus surfaces;
for each of the three aortic sinus surfaces within the geometric surface representation of the aortic sinus:
defining a replacement leaflet shape based on the surface representation of the aortic sinus;
creating a first template form presenting a concave surface corresponding to the replacement leaflet shape; and
creating a second template form presenting a convex surface corresponding to the replacement leaflet shape, wherein the concave surface and the convex surface are matably congruent;
sandwiching a substantially flat piece of prosthetic leaflet material between the concave surface of the first template form and the convex surface of the second template form, such that a first portion of the prosthetic leaflet material is secured between the concave and convex surfaces, and a second portion of the prosthetic leaflet material extends beyond the concave and convex surfaces;
trimming and discarding the second portion of the prosthetic leaflet material extending beyond the concave and convex surfaces;
separating the first template form and second template form to release the first portion of the prosthetic leaflet material, the first portion of the prosthetic leaflet material now configured in the form of a custom replacement aortic valve leaflet;
constructing an anchoring frame based on the custom replacement aortic valve leaflets; and
joining the three custom replacement aortic valve leaflets with the anchoring frame to form a replacement aortic valve prosthesis.

5. The aortic valve leaflet of claim 3 wherein the substantially flat piece of prosthetic leaflet material is a biological tissue.

6. The aortic valve prosthesis claims 4 wherein the substantially flat piece of prosthetic leaflet material is a biological tissue.

7. The aortic valve leaflet of claim 5 wherein the biological tissue comprises porcine tissue.

8. The aortic valve prosthesis of claim 6 wherein the biological tissue comprises porcine tissue.

9. The aortic valve leaflet of claim 5 wherein the biological tissue comprises bovine tissue.

10. The aortic valve prosthesis of claim 6 wherein the biological tissue comprises bovine tissue.

11. The aortic valve leaflet of claim 5 wherein the biological tissue comprises human tissue.

12. The aortic valve prosthesis of claim 6 wherein the biological tissue comprises human tissue.

13. The aortic valve leaflet of claim 3 wherein the substantially flat piece of prosthetic leaflet material comprises a synthetic material.

14. The aortic valve prosthesis of claim 4 wherein the substantially flat piece of prosthetic leaflet material comprises a synthetic material.

15. The aortic valve leaflet of claim 13 wherein the synthetic material is selected from the group of a polymer or woven fabric.

16. The aortic valve prosthesis of claim 14 wherein the synthetic material is selected from the group of a polymer or woven fabric.

17. The aortic valve leaflet of claim 13 wherein the synthetic material is selected from the group of silicone, polyurethane, polycarbonate urethane, and polytetrafluoroethylene.

18. The aortic valve prosthesis of claim 14 wherein the synthetic material is selected from the group of silicone, polyurethane, polycarbonate urethane, and polytetrafluoroethylene.
